(19) United States
(12) Patent Application Publication
(10) Pub. No.: US 2013/0189643 A1
(43) Pub. Date: Jul. 25, 2013

(54) Dental Needle and Sliding-Sheath Capping System

(71) Applicant: Michael Infanger, Chicago, IL (US)

(72) Inventor: Michael Infanger, Chicago, IL (US)

(21) Appl. No.: 13/214,474

(22) Filed: Jan. 9, 2012

(51) Int. Cl. A61M 5/32

(52) U.S. Cl. CPC A61M 5/3243
USPC 433/90

(57) ABSTRACT

An improved safety needle cap for disposable needles used on dental syringes, or on syringes of similar design, that may be removed and replaced without risk of a needle stick injury. This is accomplished by fitting a needle cap to the needle hub so that it can slide down over the syringe body and lock in place while in use. The cap will then slide back over the needle and lock in place, covering the needle cannula while not in use. In this way the user's digits are continuously below the needle, decreasing the chance for puncture. The device will use a single cap that will cover both cannulas of a double sided needle, rather than the dual cap system commonly used.

This invention is a safety device intended to reduce the risk of exposure of medical professionals to blood borne pathogens due to accidental needle stick injury incurred while attempting to recap hypodermic syringes or needles, particularly of the type used in dental offices.

### DISCUSSION OF THE PRIOR ART

In providing medical care for dental patients anesthesia is routinely necessary. Anesthesia allows dentists to perform complex medical procedures while the patient is in a sedated state allowing the dentist more freedom to operate, and minimizing the pain felt by the patient. Anesthesia is usually administered via a non-disposable syringe, through a disposable needle.

The syringes used to administer local anesthetics in dental offices and other similar syringes use a disposable cartridge of local anesthetic and a disposable needle which attaches to the front of the syringe. The disposable needle is supplied by the manufacturer with a two part protective cover. The back part of the cover fits over the mounting hub of the needle and 1 centimeter of the back end of the front cover and is removed when the syringe is loaded for use. The front portion remains as a protective cover to preserve the sterility of the needle and to protect the users while handling the syringe prior to, and after, use. The front portion of the protective cover is referred to as a “needle cap”. Because of the manner in which the back portion of the cover telescopes over the front portion and the two are sealed together, an annular ridge or shoulder is created 1 centimeter from the back end of the front portion of the cap. All commercially available needles for dental office use at this time have a similar ridge or shoulder as the apparatus used to attach the needle to most dental syringes is the same. The needles and their protective needle caps intended for use in dental offices are supplied in different lengths for use in Mandibular and Maxillary injections. The Mandibular needle cap is 4.5 to 5 centimeters long from the previously mentioned shoulder to the front end and the Maxillary needle cap is 3 to 3.5 centimeters long between those points. The diameter of the cap at the shoulder is 1 centimeter and immediately behind the shoulder the diameter is less, usually 0.85 centimeters.

The needle cap is removed immediately prior to administering the injection to the patient. The danger of an infectious needle stick occurs when the protective needle cap is replaced on the needle, post injection, which is now contaminated with the patient's blood and saliva. A single method dominates the dental field and it consists of a two handed process. The syringe is normally held in the operator's favored hand and the needle cap is held in the other. The cap and needle are then slowly brought together anti! the cap has sufficiently covered the needle and locked into place at the junction of the needle and syringe. If the needle misses the opening in the cap, there is a significant chance that the hand holding the cap will be stuck and the operator exposed to any blood-borne infection carried by the patient. To avoid this problem, the Center for Disease Control currently recommends discarding disposable syringes without replacing the needle cap. This is impractical in the dental office because only the needle, its covers, and the anesthetic capsule (or cartridge) are disposable; the remainder of the syringe is sterilized and reloaded. Often a needle is used multiple times on a single patient to administer additional anesthetic. Though the risk of an accidental stick is greater handling the uncapped syringe needle than the risk to recap the needle, needles are still reused to save time and money.

There are techniques for handling the recapping procedure to avoid the danger of a stick such as the “scooping” the cap off a table top, commonly called the ‘one-handed scoop method’ with the needle and pressing the cap against a wall to seat the cap on the needle base or holding the cap with a hemostat or forceps instead of the hand. These techniques work, but they are awkward at best, allow for the risk of picking up spatter which might have fallen onto the dental tray and often ignored out of convenience. Moreover the method name is a misnomer; it in fact does require a second hand to fasten the cap securely to the needle hub once it is in place. While this method is commonly taught in dental schools, it is rarely used by tenured dentists in factor of the two-handed technique.

### DRAWINGS

FIG. 1 depicts a cross section of one embodiment of safety device 1 according to the present invention.

FIG. 2 depicts a side view of one embodiment of safety device 1 according to the resent invention.

FIG. 3 depicts an angled view of one embodiment of safety devices 1 according to the resent invention.

FIG. 4 depicts a head-on view of the invention, showing the slot in the cap head

FIG. 5 depicts a diagram of the use of the device

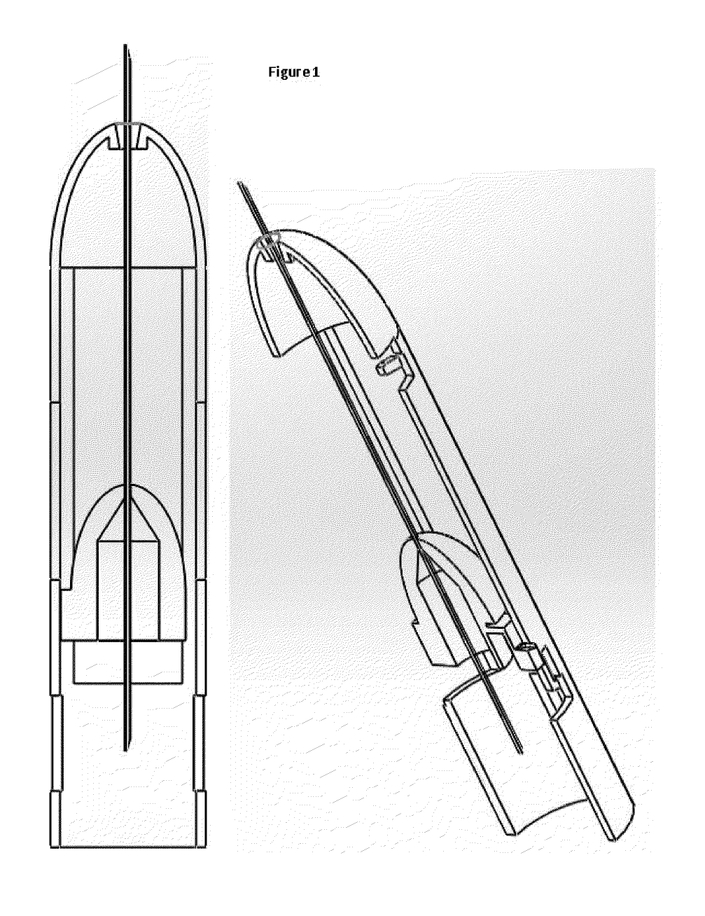

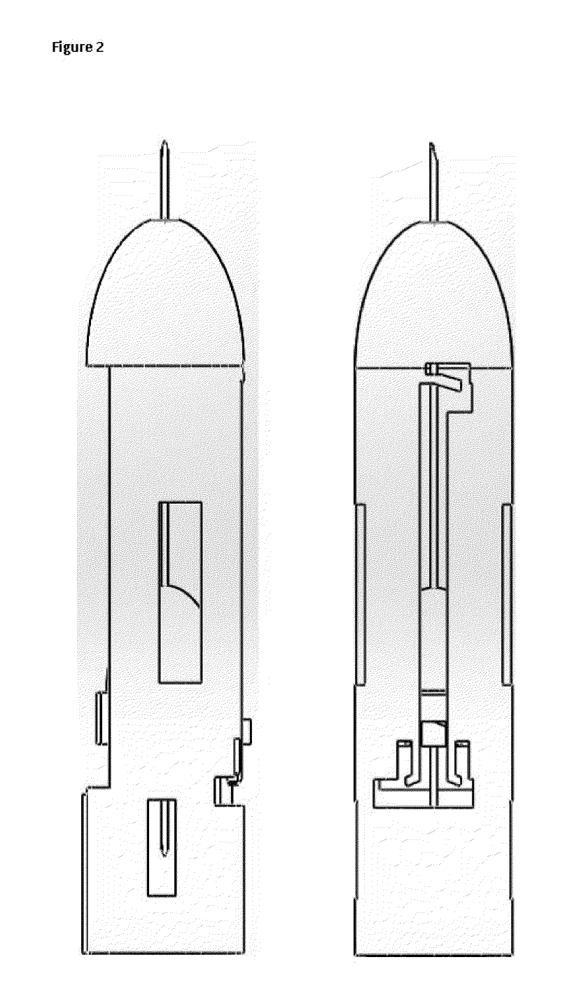

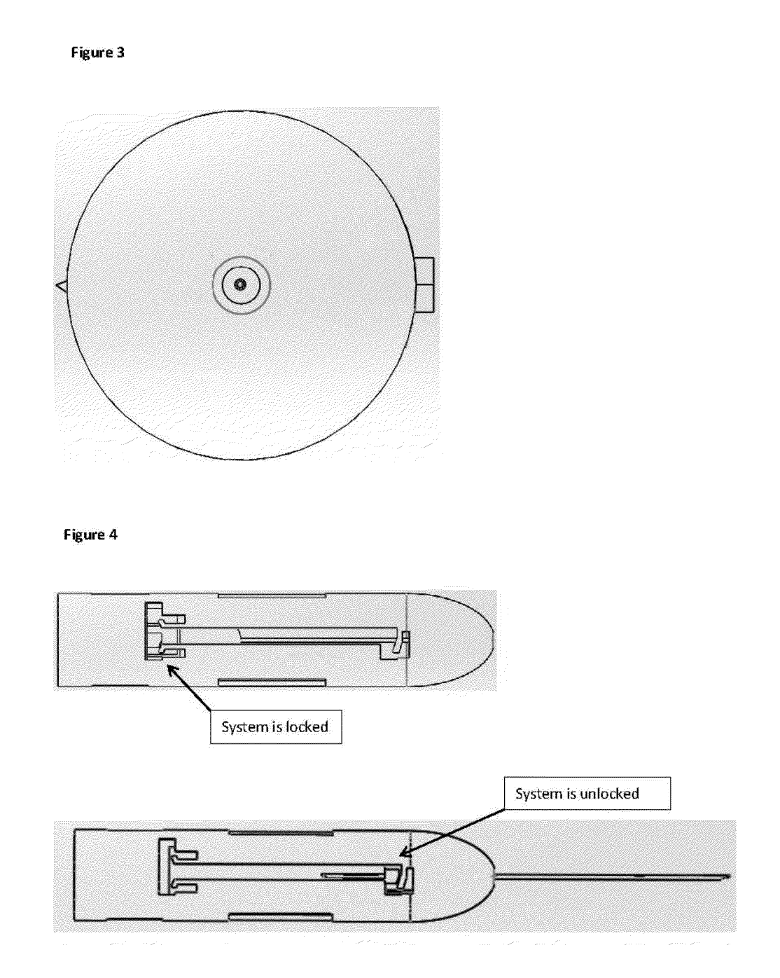

### SUMMARY OF THE INVENTION

A safety device is provided for recapping exposed needles safely, reducing the risk of injury to medical professionals. The safety device comprises a plastic cap having a slot at the head, a large opening at the foot and a slot along the side into which the needle hub will slide. The cap is attached to the needle hub and cannot be removed unless broken. The cap can be slid down over the syringe while the needle is in use and slid back over the needle once it has been contaminated with fluid. The distal end of the contaminated needle will enter the device through the opening, and rest onto the needle holder. From there the needle is allowed to slide up the needle holder until the distal end of the needle has reached the needle shield. Once the needle reaches the needle shield it is encased within the shield thus preventing it from accidentally infecting the dental professional.

### DESCRIPTION

In the preferred embodiment a cartridge of medicament is loaded into a dental syringe. The dental syringe consists of a hollow cylindrical body section having an open proximal and distal end, a plunger, a compartment located on the proximal end of the hollow cylindrical body intended to receive a medicament cartridge, and a fitting on the proximal end of both the hollow cylindrical body, and the medicament cartridge chamber intended to house double ended needle assembly. The syringe in this embodiment may correspond to or be adapted from, for example, those of U.S. Pat. No. 6,605,744 “Injection Apparatus and Method of Injecting,” issued Sep. 20, 1971 by E. M. Dwyer; U.S. Pat. No. 3,820,652 “Packaged Syringe Construction,” issued Jun. 28, 1974 by Thackston. Both of which care herein incorporated by reference to the extent that is non-contradictory herewith.

The double ended needle cannula described above is shipped completely sheathed by two interlocking plastic caps, once cap for each cannula, intended to preserve the sterility of the needle cannula assembly. The distal protective cap is to be removed, unsheathing the distal needle cannula. The double ended needle assembly is then to be inserted into it's fitting on the dental syringe by placing the exposed distal cannula first allowing it to enter the medicament cartridge compartment and pierce the elastomeric stopper of the medicament vial itself. The needle assembly comprises an interior bore that provides fluid communication across the length of the needle cannula, such that fluid from a medicament cartridge introduced at the distal end can pass through the length of the assembly and exit through the proximal end into the intended recipient (tissue), typically in response to an increase in pressure. This double ended needle cannula may be adapted from those described in U.S. Pat. No. 5,222,502, “Blood Collecting Needle,” issued Apr. 19, 1994 to K. Kurose, herein incorporated by reference to the extent that it is non-contradictory herewith.

Once the double ended needle assembly is in place and has sufficiently pierced the elastomeric stopper of the medicament cartridge, and is in fluid communication with the medicament itself the cap on the proximal end of the needle assembly may be removed exposing the proximal needle cannula. The bevel of the proximal needle cannula is then to be inserted into the desired tissue allowing for fluid to traverse the length of the needle cannula assembly and come into contact with the tissue. The medical professional may then depress the plunger of the dental syringe allowing for the pressure within the medicament cartridge to increase, thus delivering a pre-determined amount of medicament through the needle assembly into the desired tissue. This procedure may be repeated as many times as deemed necessary by the medical professional to ensure proper dosage for the patient. Once this procedure has been completed the bevel of the proximal needle cannula is removed from the patient's tissue. This procedure may also be used to deliver medicament into the patient's blood stream by substituting “interior of vein” for “tissue.”

Upon completion of this procedure the now contaminated proximal needle cannula must be re-sheathed (re-capped) in order to prevent accidental needle stick injury to the dental professional or assistants. In order to successfully accomplish this goal the following method must be utilized.

The dental professional will grip the cap body with the thumb and second knuckle of the forefinger. The professional will then twist to his left so that the needle is released and the hub is sitting in the hub guide. The professional then slides the cap up the hub guide until the hub reaches the end of the guide. The professional finally twists the cap to the right so that the needle again locks in place.

1) A protective cover for a syringe unit, the protective cover comprising
(a) Housing
(b) Slot for cannula head
(c) Sheathed locking mechanism upper locking mechanism
(d) Unsheathed locking mechanism lower locking mechanism
(e) Hub guide
(f) Fitted Sheath
(g) Finger grips on sheath

2) The device of claim 1 with at least one opening in the housing to allow for a needle to be inserted

3) The device of claim 1 with at least one fitted sheath

4) The device of claim 1 with at least 1 finger grip

5) The device of claim 1 wherein sheath can bend needle back when retracted

6) The device of claim 1 wherein the slot at the tip of the cap is smaller than a finger tip

7) The device of claim 1 wherein the sheath exposes all of the needle when slid down the hub guide

8) The device of claim 1 wherein the sheath covers all of the needle when slid up the hub guide
